(19) United States
(12) Patent Application Publication
(10) Pub. No.: US 2011/0189296 A1
(43) Pub. Date: Aug. 4, 2011

(54) BONE SUBSTITUTE CONTAINING POROUS BIO-GLASS AND CALCIUM SULPHATE

(75) Inventors: André Rauïs, Bruxelles (BE); Rachid Zenati, Neuville sur Saone (FR)

(73) Assignee: NORAKER, Villeurbanne (FR)

(21) Appl. No.: 13/060,958

(22) PCT Filed: Aug. 24, 2009
(86) PCT No.: PCT/EP09/60874

§ 371 Date: Apr. 19, 2011

(30) Foreign Application Priority Data
Aug. 27, 2008 (BE) 2008/0474

(51) Int. Cl. A61K 9/00; A61K 33/06; A61P 43/00

(52) U.S. Cl. 424/489; 424/696

(57) ABSTRACT

The invention relates to a composition for a bone substitute that includes powdery or populated porous bio-glass and powdery alpha hemihydrate calcium sulphate. The invention also relates to an injectable composition for forming a bone substitute that includes said composition of powdery or granulated porous bio-glass and powdery alpha hemihydrate calcium sulphate, with 10 to 50 wt % of added water.

The present invention relates to a new chemical composition for a bone substitute, composed of a mixture of porous bioglass and calcium sulfate.

Bone defects encountered in orthopedic surgery and in dentistry have been treated with bone grafting for some time. The graft is either taken from the patient (autograft), which involves an additional surgical intervention, or obtained from a human tissue bank (allograft), which is associated with higher costs and risks of contamination. There have also been endeavors to use chemical bone substitutes in surgery for some time.

For more than a century, calcium sulfate (“plaster of Paris”) has been used to correct bone defects (for filling osteitis cavities and vertebral abscesses in osseous tuberculosis, etc.) with noteworthy results. Calcium sulfate supplies the necessary material (Ca++) and scaffold for new bone growth. Nevertheless, it has two disadvantages: it is too rapidly resorbed and does nothing to stimulate bone growth.

There are other chemical substitutes currently in use, for example:

HAP (hydroxyapatite)

Biphasic HAP

HAP carbonate

Beta-TCP (biphasic calcium phosphate)

DCPD (dicalcium phosphate dihydrate)

HAP+silicon

HAP+collagen

Calcium sulfate

Calcium sulfate+PLLA

Bioglass (“Bioglass”®)

Porous bioglass

and possible combinations of these products.

Some of these products and chemical compounds (HAP, beta-TCP, and DCPD) have the disadvantage of being very slowly resorbed by the body and thus their presence prevents new bone growth.

Bioglass is resorbable. The resorption time varies inversely with the contact surface. Hence porous bioglass, which exhibits a substantial contact surface with body fluids, will be rapidly resorbed compared to unrefined bioglass.

Of the 47 commercially available bone substitutes surveyed by the AAOS (American Academy of Orthopedic Surgeons, January 2008), none correspond to the ideal bone substitute.

Other reference articles in this field include:
Kelly, C. M. et al., “The Use of a Surgical Grade Calcium Sulfate as a Bone Graft Substitute,” Clin. Orthop. Rel. Res. 382, 2001, pp. 42-50.
Peltier, L. F., “The Use of Plaster of Paris to Fill Defects in Bone,” Clin. Orthop. Rel. Res. 21, 1961, pp. 1-31.
Alexander, D. et al., “Efficacy of Calcium Sulfate Plus Decompression Bone in Lumbar and Lumbosacral Spinal Fusion: Preliminary Results in 40 patients,” Can. J Surg. 44(4), 2001, pp. 262-266.

### Schepers et al., “Bioactive Glass Particles on Narrow Size Range: A New Material for the Repair of Bone Defects,” Implant Dent. 1993, 2, 151-156.

It can thus be stated that the required characteristics of an ideal bone substitute are:
1. provision of bone material (Ca++)
2. provision of a scaffold for osteoconduction, i.e., a substrate for new bone growth.
3. being resorbable within a suitable time interval so that its presence will not prevent new bone growth.
4. provision of osteoinduction, i.e., a stimulation of new bone growth by promoting the development of bone cells.
5. being malleable and even injectable with a syringe or trocar during the preparation phase.
6. provision of rapid mechanical anchoring (rapid setting) with a mechanical resistance to compression equivalent to that of cancellous bone.

Of the chemical products suitable for implantation in the body of a mammal (human being, among others), mention can be made of:
1. Bioglass (SiO₂, CaO, Na₂O, P₂O₅) or (“Bioglass 45S5”®):
   Osteostimulative
   Source of calcium and phosphorus
   But slowly resorbed, and non-malleable.
2. Porous bioglass (as defined in the documents WO 2006/018531 A2 and US 2008/0038534 A1 of the INSA) or as defined in the documents US 2007/0162151 A1, U.S. Pat. No. 5,648,301 A, U.S. Pat. No. 5,676,720 B1, U.S. Pat. No. 6,406,498 A, and U.S. Pat. No. 6,413,538 B1)
   Owing to the size of its pores, it provides osteoconduction for new bone cell growth.
   It is strongly osteoinductive.
   Owing to its large contact surface with body fluids, it is more rapidly resorbed than compact bioglass.

Medical grade calcium sulfate (CaSO₄, “plaster of Paris”) has been in use for over a century as a bone substitute for correcting bone defects, with noteworthy results. It provides the calcium needed for reconstructing bone, it is osteoconductive, malleable, and injectable, provides rapid mechanical anchoring (sets rapidly), and is rapidly resorbed (in 4 to 8 weeks). It is currently in widespread use and sold as a bone substitute under the trade names
Surgiplaster (Orthogen Corporation)
Calceon 6 (Synthes USA)
Ceraplast (Ceraver)
Surgical plaster (ACE CO)
Osteoset (Wright Medical Technologies)
MIIG 115 (Wright Medical Technologies)
MIIG×3 (Wright Medical Technologies), some of which are the object of patents:
U.S. Pat. No. 5,614,206
U.S. Pat. No. 5,807,567
U.S. Pat. No. 6,030,636
U.S. Pat. No. 6,652,887

Theoretically, the two chemical products (bioglass and calcium sulfate) are complementary; hence mixing them should give rise to an ideal bone substitute as defined above. However, the mixing and preparation of unrefined bioglass granules and plaster of Paris turns out to be unsuitable because CaSO₄ will not set in the presence of unrefined bioglass.

In contrast, it has been shown that porous bioglass obtained from unrefined bioglass as defined in the document WO 2006/018531 A2, in granular form and under certain conditions, is capable of forming a mixture with calcium sulfate that is injectable, sets rapidly, and provides excellent mechanical resistance.

The present invention relates to such a composition of porous bioglass granules and calcium sulfate, and more particularly to a powdery composition for a bone substitute comprising powdery or granular porous bioglass and powdery calcium sulfate alpha-hemihydrate.

According to a preferred characteristic of the invention, the composition comprises 0.25 to 5 parts by weight of calcium sulfate alpha-hemihydrate per 1 part of bioglass.

According to another preferred particularity of the invention, the particle size distribution of the porous bioglass is in the range of between 0 and 1,000 microns (more particularly between 1 and 1,000 microns).

The first material, porous bioglass (such as that supplied by the company “Noraker”), may be present in the form of granules of variable sizes, namely:
Particle size distribution ranging from 1 to 90μ.
Particle size distribution ranging from 90 to 200μ.
Particle size distribution ranging from 200 to 300μ.
Particle size distribution ranging from 300 to 900μ.
and other desirable dimensions.

According to the invention, the size of the porous bioglass granules is critical.

Granules from 1 to 90μ in size result in too slow a setting of the mixture. On the other hand, granules 900μ in size result in good setting of the mixture, but detract from injectability due to their excessively large size.

According to a preferred characteristic of the invention, the ideal size is in the range of between 100 and 300μ.

The second material, calcium sulfate hemihydrate, is present in the form of a fine powder which, when mixed with water, chemically reacts to form calcium sulfate dihydrate, which exhibits particularly high mechanical resistance.

Calcium sulfate hemihydrate occurs in two crystalline forms: alpha-hemihydrate and beta-hemihydrate. When mixed with water, the alpha form will produce a more solid dihydrate than the beta form and will be resorbed more slowly in vivo (around 30 days for the beta form vs. 50 days for the alpha form).

Hence it is imperative to choose an alpha-hemihydrate for the composition of the invention. The latter may be obtained from the dihydrate either by a special heating process or by a chemical process (dehydration of the dihydrate by heating in diluted sulfuric acid solution).

An alpha-hemihydrate powder is sold under the trade name CAPSET® by the company Lifecore (Chaska, Minn.).

In order for setting to occur rapidly, it is highly preferable to add a set accelerator. According to a preferred characteristic of the invention, the composition thus comprises at least one set accelerator chosen from among:
NaCl
finely-ground dihydrate (particle size <50μ) at a dose varying from 1 to 15 wt % in proportion to hemihydrate. At any rate, the presence of dihydrate is required in order for the mixture to set in a protein medium.

Preference is given to the calcium sulfate dihydrate having a particle size distribution ranging from 10 to 100 microns.

The company Lifecore (Chaska, Minn.) markets a pre-loaded alpha-hemihydrate under the trade name of CAPSET®.

The powdery composition of the invention preferably comprises 1 to 15 wt % calcium sulfate dihydrate in proportion to calcium sulfate alpha-hemihydrate, and more particularly
20 to 80 wt % powdery or granular porous bioglass,
20 to 80 wt % calcium sulfate alpha-hemihydrate,
1 to 15 wt % calcium sulfate dihydrate with, optionally, 0 to 2 wt % NaCl.

The porous bioglass used preferably has a porosity (measured by a geometric method) of 50 to 80%, with macropores having a mean diameter of 100 to 1250 microns and micropores having a mean diameter ≦5 microns, and preferably has a content of 40 to 55 wt % SiO₂, 15 to 25 wt % CaO, 15 to 25 wt % Na₂O, and 1 to 9 wt % P₂O₅.

A preferred powdery composition of the invention comprises:
1. 20 to 80 wt % granular porous bioglass (10 to 900μ in size),
2. 20 to 80 wt % powdery calcium sulfate alpha-hemihydrate,
3. 1 to 15 wt % calcium sulfate dihydrate (fine powder),
4. optionally, 0 to 2 wt % NaCl,
5. and optionally, 0 to 2 wt % hydroxypropyl methylcellulose.

The mixture can be prepared as follows: the CaSO₄ alpha-hemihydrate and CaSO₄ dihydrate powders and the porous bioglass granules are dry-mixed in a vessel at ambient temperature. NaCl and/or hydroxypropyl methylcellulose is optionally added to the mixture.

The invention also relates to a composition for an injectable bone substitute comprising a powdery composition as described above, mixed with deionized water, at ambient temperature. Preference is given to mixing the preparation for thirty seconds.

A preferred composition for an injectable bone substitute of the invention comprises:
1. 20 to 80 wt % granular porous bioglass (10 to 900μ),
2. 20 to 80 wt % powdery calcium sulfate alpha-hemihydrate,
3. 1 to 15 wt % calcium sulfate dihydrate (fine powder),
4. a suitable volume (preferably 10 to 50 wt % of the dry mixture) of deionized water,
5. optionally, 0 to 2 wt % NaCl,
6. and optionally, 0 to 2 wt % hydroxypropyl methylcellulose.

The setting of the CaSO₄ alpha-hemihydrate/CaSO₄ dihydrate/porous bioglass granules/deionized water mixture, with or without added NaCl and/or hydroxypropyl methylcellulose, takes place in three phases:
After the constituents are mixed, the composition becomes plastic and injectable and remains so for several minutes.
The next phase is the Initial Setting Time (IST), after which the composition is no longer malleable or injectable, but still has not reached compression strength.
An additional interval (of 5 to 15 min.) leads to a final setting (Final Setting Time=FST) with a resistance to compression of around 1 MPa, which is equivalent to that of porotic cancellous bone. The resistance attains 8 MPa after one hour.

The optionally added hydroxypropyl methylcellulose acts as a plasticizer capable of improving the injectability of the mixture. The NaCl can be used as a supplementary set accelerator.

The bone substitute compositions according to the invention are injectable, osteoconductive, osteoinductive, resorbable, and exhibit a mechanical resistance comparable to that of bone.

### EXAMPLE 1

A composition for an injectable bone substitute according to the invention is achievable with:
10 grams of 100 to 300μ granular porous bioglass.
10 grams of calcium sulfate alpha-hemihydrate.
1 gram of fine dihydrate powder.
12 grams of deionized water.

The first three materials are dry-mixed, and then added to the water and mixed for 30 seconds. The mixture remains injectable for 4 minutes (until the IST). The final setting time (FST) is 10 minutes.

### EXAMPLE 2

Another composition for an injectable bone substitute according to the invention is achievable with:
10 grams of 100 to 300μ granular porous bioglass.
20 grams of calcium sulfate alpha-hemihydrate.
2 grams of calcium sulfate dihydrate.
0.1 gram of NaCl.

The mixture remains injectable for 6 minutes and final setting occurs after 17 minutes.

### EXAMPLE 3

A composition for an injectable bone substitute according to the invention is achievable as in example 2, with 1 gram of hydroxypropyl methylcellulose added.

The viscosity of the mixtures of examples 1-3 during the preparation phase prior to the initial setting (IST) is such that said mixtures are injectable with a syringe or capable of being manipulated like finishing putty.

The weak exothermic reaction (<40° C.) that takes place as the mixture sets allows the incorporation of pharmaceutical agents in the mixture. Examples thereof include antibiotics (gentamicin, tobramycin, cephalosporins, vancomycin, etc.), chemotherapeutic agents (cis-platinum, methotrexate, isofosfamide, etc.), analgesics (lidocaine, etc.).

The two main constituents of the composition will exhibit different resorption rates after implantation in the body of a mammal (human being, among others).

Calcium sulfate is resorbed first, disappearing in approximately 50 days, and this resorption gradually allows the porous bioglass to come into contact with the tissues. Bioglass is more slowly resorbed, thus allowing it to exert its osteostimulation effect. The graduated resorption of the two constituents thus takes place at a rate corresponding to the speed of new bone growth.

1. Powdery composition for a bone substitute containing porous bioglass, characterized in that said composition comprises powdery or granular porous bioglass and powdery calcium sulfate alpha-hemihydrate.

2. Composition as in claim 1, characterized in that said composition comprises 0.25 to 5 parts calcium sulfate alpha-hemihydrate to 1 part bioglass.

3. Composition as in claim 1, characterized in that the particle size distribution of the porous bioglass is in the range of between 0 and 1,000 microns.

4. Composition as in claim 1, characterized in that said composition comprises at least one set accelerator chosen from finely-ground calcium sulfate dihydrate and sodium chloride.

5. Composition as in claim 4, characterized in that the calcium sulfate dihydrate has a particle size distribution in the range of between 10 and 100 microns.

6. Composition as in claim 4, characterized in that said composition comprises 1 to 15 wt % calcium sulfate dihydrate in proportion to calcium sulfate alpha-hemihydrate.

7. Composition as in claim 4, characterized in that said composition comprises
20 to 80 wt % powdery or granular porous bioglass,
20 to 80 wt % calcium sulfate alpha-hemihydrate,
1 to 15 wt % calcium sulfate dihydrate, and optionally, 0 to 2 wt % NaCl.

8. Composition as in claim 1, characterized in that the porous bioglass has a porosity (measured by a geometric method) of 50 to 80%, with macropores having a mean diameter of 100 to 1,250 microns and micropores having a mean diameter≦5 microns.

9. Composition as in claim 1, characterized in that the porous bioglass has a content of 40 to 55 wt % SiO₂, 15 to 25 wt % CaO, 15 to 25 wt % Na₂O, and 1 to 9 wt % P₂O₅.

10. Composition for an injectable bone substitute comprising a mixture of water and a powdery composition as in claim 1.

11. Composition for an injectable bone substitute, characterized in that said composition comprises 10 to 50 wt % water in proportion to the amount of dry matter.

12. Composition for an injectable bone substitute as in claim 10, characterized in that said composition comprises 0.1 to 2 wt % NaCl.
